(19) United States
(12) Patent Application Publication
(10) Pub. No.: US 2024/0075144 A1
(43) Pub. Date: Mar. 7, 2024

(54) Interleukin-37, Chimeric Antigen Receptors, Nucleic Acids, and Vectors Encoding the Same and Uses in Cancer Therapies

(71) Applicants: Emory University, Atlanta, GA (US); Children's Healthcare of Atlanta, Inc., Atlanta, GA (US)

(72) Inventors: Curtis J. Henry, Atlanta, GA (US); Sunil S. Raikar, Decatur, GA (US); Sarwish Rafiq, Brookhaven, GA (US)

(21) Appl. No.: 18/271,364

(22) PCT Filed: Jan. 7, 2022
(86) PCT No.: PCT/US2022/011618

§ 371 Date: Jul. 7, 2023

Related U.S. Application Data
(60) Provisional application No. 63/135,218, filed on Jan. 8, 2021.

(51) Int. Cl. A61K 39/00; A61P 35/02; C07K 14/54; C07K 14/55; C07K 14/705; C07K 14/725; C07K 16/28; C12N 15/86

(52) U.S. Cl. CPC A61K 39/464412; A61K 39/4611; A61K 39/4631; A61K 39/4635; A61K 39/4636; A61P 35/02; C07K 14/54; C07K 14/55; C07K 14/7051; C07K 14/70517; C07K 14/70521; C07K 16/2803; C12N 15/86; A61K 2239/38; A61K 2239/39; A61K 2239/48; C07K 2317/622; C07K 2319/02; C07K 2319/03; C07K 2319/50; C12N 2740/15043

(57) ABSTRACT

This disclosure relates to therapeutics containing IL-37, chimeric antigen receptors, nucleic acids, or vectors encoding the same. In certain embodiments, this disclosure relates to methods of treating cancer comprising administering a nucleic acid or vector encoding interleukin-37 to a subject diagnosed with cancer and administering T cells expressing a chimeric antigen receptor to the subject. In certain embodiments, this disclosure relates to methods of treating cancer comprising administering a nucleic acid or vector encoding interleukin-37 and a chimeric antigen receptor to a subject diagnosed with cancer.

### CROSS-REFERENCE TO RELATED APPLICATIONS

This application claims the benefit of U.S. Provisional Application No. 63/135,218 filed Jan. 8, 2021. The entirety of this application is hereby incorporated by reference for all purposes.

### STATEMENT REGARDING FEDERALLY SPONSORED RESEARCH OR DEVELOPMENT

This invention was made with government support under grant numbers CA160798, CA248962, and HD072245 awarded by the National Institutes of Health. The government has certain rights in this invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 21009PCT_ST25.txt. The text file is 25 KB, was created on Jan. 7, 2022, and is being submitted electronically via EFS-Web.

### BACKGROUND

Declining immunity is a hallmark of aging in mice and humans. The effect of a waning immune response with age is thought to contribute to increased infection-related mortalities in the elderly and higher cancer incidence. Thus, there is a need to identify improved methods for treating subjects as they age. Chronic inflammation may be one cause underlying aging-associated immune impairments. “Inflammaging” in mice and humans is characterized by a systemic increase in pro-inflammatory cytokines including tumor necrosis factor-alpha (TNF-α), interleukin-6 (IL-6), interleukin-1 beta (IL-1β), and C-reactive protein (CRP). Elevated levels of these inflammatory mediators have been shown to regulate the homeostasis and function of hematopoietic stem, progenitor, and mature immune cells, which express cytokine receptors that regulate their steady and activated states.

Antibody-mediated and chimeric antigen receptor (CAR) T-cell therapies have shown remarkable success in treating previously intractable diseases such as melanoma. Immunotherapies are also used to treat relapsed and refractory hematological malignancies including B-cell acute lymphoblastic leukemia (B-ALL) and diffuse large B-cell lymphoma (DLBCL). Despite the success of immunotherapies in patients with terminal disease, a substantial segment of patients receiving CD19-directed CAR T-cell therapy relapse within the first 2 years of receiving treatment. Thus, there is a need to identify improved therapies.

Henry et al. report aging-associated inflammation promotes selection for adaptive oncogenic events in B cell progenitors and that the anti-inflammatory cytokine interleukin-37 (IL-37) reduces aging-associated inflammation and improves hematopoiesis in aged mice. Journal of Clinical Investigation, 2015, 125(12), 4666-4680.

Ji et al. report exogenous interleukin 37 ameliorates atherosclerosis via inducing the Treg response in ApoE-deficient mice. Scientific Reports, 2017, 7(1), 3310.

Liu et al. report IL-37 suppresses hepatocellular carcinoma growth by converting pSmad3 signaling from JNK/pSmad3L/c-Myc oncogenic signaling to pSmad3C/P21 tumor suppressive signaling. Oncotarget, 2016, 7(51), 85079-85096

Zhao et al., report IL-37 mediates the antitumor activity in hepatocellular carcinoma. Scientific Reports, 2014, 4, 5177.

References cited herein are not an admission of prior art.

### SUMMARY

This disclosure relates to therapeutics containing IL-37, chimeric antigen receptors, nucleic acids, or vectors encoding the same. In certain embodiments, this disclosure relates to methods of treating cancer comprising administering a nucleic acid or vector encoding interleukin-37 to a subject diagnosed with cancer and administering T cells expressing a chimeric antigen receptor to the subject.

In certain embodiments, this disclosure relates to methods of treating cancer or a hematological malignancy comprising administering an effective amount of a recombinant IL-37 protein or a nucleic acid encoding IL-37 to a subject in need thereof. In certain embodiments, the subject is over 55 or 65 years of age.

In certain embodiments, the nucleic acid encoding IL-37 or recombinant IL-37 protein is administered during, after, or more than one day before administration of autologous or non-autologous T cells optionally comprising a chimeric antigen receptor. In certain embodiments, the T cells are contacted with to anti-CD3 and anti-CD28 antibodies providing activated T cells prior to administration.

In certain embodiments, this disclosure relates to methods of treating cancer or a hematological malignancy comprising administering an effective amount of T cells expressing a cancer targeting chimeric antigen receptor and administering a vector encoding a recombinant IL-37 protein to a subject in need thereof. In certain embodiments, the T cells are contacted with anti-CD3 and anti-CD28 antibodies providing activated CAR T cells prior to administration.

In certain embodiments, this disclosure relates to methods of treating cancer or a hematological malignancy comprising administering an effective amount of T cells expressing a cancer targeting chimeric antigen receptor and expressing a recombinant IL-37 protein to a subject in need thereof. In certain embodiments, the subject is over 55 or 65 years of age. In certain embodiments, the cancer targeting chimeric antigen receptor and the recombinant IL-37 protein are expressed in a single vector. In certain embodiments, the cancer targeting chimeric antigen receptor and the recombinant IL-37 protein are expressed in separate vectors.

In certain embodiments, this disclosure relates to methods of treating cancer or a hematological malignancy comprising administering an effective amount of T cells expressing a cancer targeting chimeric antigen receptor in combination with a nucleic acid encoding IL-37 or a recombinant IL-37 protein to a subject in need thereof. In certain embodiments, the subject is over 55 or 65 years of age.

In certain embodiments, the methods reported herein are done in combination with administering another anticancer agent to the subject. In certain embodiments, the nucleic acid encoding IL-37 is administered in combination with another anticancer agent. In certain embodiments, administrating T cells expressing a nucleic acid encoding IL-37 is administered in combination with another anticancer agent. In certain embodiments, the T cell are expressing a chimeric antigen receptor. In certain embodiments, the anticancer agent is a checkpoint inhibitor, an anti-PD-1, anti-PD-L1 anti-CTLA4 antibody or combinations thereof. In certain embodiments, the anti-CTLA4 antibody is ipilimumab or tremelimumab. In certain embodiments, the anti-PD1 antibody is nivolumab, pembrolizumab, or cemiplimab. In certain embodiments, the anti-PD-L1 antibody is atezolizumab, avelumab, or durvalumab.

In certain embodiments, the subject to be treated has or is diagnosed with a hematological malignancy and has previously received a bone marrow or hematopoietic stem cell transplant, e.g., wherein stem cells are collected from the bloodstream or bone marrow.

In certain embodiments, the nucleic acid encoding IL-37 or the recombinant IL-37 protein or T cells optionally expressing a chimeric antigen receptor are administered to a subject with a lymphodepleted environment due to prior or concurrent administration of a lymphodepleting agent. In certain embodiments, the of lymphodepleting agent is cyclophosphamide, fludarabine, or combination thereof.

In certain embodiments, the hematological malignancy is selected from leukemia, acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), chronic myeloid leukemia (CML), B-cell acute lymphoblastic leukemia (B-ALL), myeloproliferative neoplasms (MPNs), and lymphomas, Hodgkin's lymphomas, and non-Hodgkin's lymphomas such as Burkitt lymphoma, B-cell lymphoma, or diffuse large B-cell lymphoma (DLBCL).

In certain embodiments, this disclosure relates to vectors encoding a chimeric antigen receptor and IL-37. In certain embodiments, the chimeric antigen receptor and IL-37 are separated by a self-cleaving spacer, e.g., GSGATNFSLLKQAGDVEENPGP (SEQ ID NO: 21). In certain embodiments, IL-37 is connected to the N-terminal of the self-cleaving spacer and the chimeric antigen receptor is connected to the C-terminal of the self-cleaving spacer, and IL-37 has the amino acid sequence of

MSFVGENSGVKMGSEDWEKDEPQCCLEDPAGSPLEPGPSLPTMNFVHTSPKVKN LNPKKFSIHDQDHKVLVLDSGNLIAVPDKNYIRPEIFFALASSLSSASAEKGSPILLGVSK GEFCLYCDKDKGQSUPSLQLKKEKLMKLAAQKESARRPFIFYRAQVGSWNMLESAAHP GWFICTSCNCNEPVGVTDKFENRKHIEFSFQPVCKAEMSPSEVSD (SEQ ID NO: 18) or variants thereof, e.g., have greater than 60, 70, 80, 85, 90, 95, 98 or 99% sequence identity.

In certain embodiments, this disclosure relates to peptides disclosed herein and variants thereof and nucleic acids and vectors encoding the same. In certain embodiments, this disclosure relates to cells and other expression systems comprising said nucleic acids and vectors. In certain embodiments, this disclosure contemplates pharmaceutical compositions comprising peptides disclosed herein, or nucleic acids or vectors encoding peptides disclosed herein.

### BRIEF DESCRIPTION OF THE SEVERAL VIEWS OF THE DRAWINGS

FIG. 1A shows data indicating interleukin-37 suppresses inflammaging, and decreased levels are observed in aged human monocytes. Monocytes were purified from PBMCs of healthy donors using MACs selection. The gene expression levels are shown for young, middle-aged, and old donors.

FIG. 1B shows data where C57BL/6 wild-type and IL-37 transgenic mice were aged for 24 months and dissected to observe potential anatomical changes. The spleen weights are shown.

FIG. 1C shows data wherein young (2 months) and old (24 months) C57BL/6 mice were treated every other day for 2 weeks with control Ig or rIL-37, and the ratio of CD4+ to CD8+ T-cells was determined via flow cytometric analysis.

FIG. 2A shows data on naïve CD4+ T-cells and CD8+ T-cells purified from mice treated with recombinant IL-37 using MACs selection and stimulated in vitro with αCD3/αCD28. Data on day 3 post-stimulation, the mean surface expression of CD44 and PD1 (mean fluorescence intensity [MFI]). This data indicates that recombinant IL-37 treatment reduces PD-1 surface expression and improves the function of aged T-cells. Aged (24 months old) C57BL/6 mice were treated with control immunoglobulin (Control Ig) or recombinant IL-37 (rIL-37) every other day for 2 weeks.

FIG. 2B shows data on the percentage and MFI of IL-2/IFN-7-producing T-cells which were determined using flow cytometric analysis.

FIG. 3A illustrates experiments indicating recombinant IL-37 treatment protects aged mice from B-ALL pathogenesis in a T-cell dependent manner. Aged (24 months old) C57BL/6 mice were treated with T-cell depleting antibodies (αCD4 and αCD8) 2 days prior to intravenous challenge with BCR-ABL+Arf−/− murine B-ALL cells (mB-ALL). Mice were also treated with Control Ig or rIL-37 1 day prior to the injection of mB-ALL cells, and this treatment continued throughout the experiment.

FIG. 3B shows survival which was monitored for over 3 months.

FIG. 4A illustrates experiments with results indicating recombinant IL-37 treatment improves the efficacy of aged CAR T-cells. Murine CD3+ T-cells were purified from aged (24 months old) C57BL/6 wild-type mice and transduced to express CD19-directed CARs (transduced cells express GFP). Aged CAR T-cells were then injected into aged wild-type mice which were then treated with control immunoglobulin (Control Ig) or recombinant IL-37 (rIL-37) once weekly for 2 weeks. After 2 weeks of treatment, GFP+ CAR T-cells were sorted from mice and stimulated in vitro with CD19-expressing murine BALL cells. On day 3 of culture, IL-2 and IFN-7 production from aged CAR T-cells was assessed by flow cytometric analysis. NOG immunocompromised (6 months old) mice were intravenously challenged with human B-ALL cells (REH cells). On day 10 post-transplantation (when signs of morbidity were observed in all mice), mice were injected with CD19-directed CAR T-cells from an aged donor (67 years old). Mice were simultaneously injected with Control Ig or rIL-37 and this treatment was continued every 5 days until the experiment was terminated.

FIG. 4B shows survival data which was monitored for over 3 months.

FIG. 5 illustrates a bicistronic construct encoding enhanced green fluorescent protein (eGFP), which can be substituted with an IL-37 sequence, and a CD19-CAR. The transgene includes a 5′ long terminal repeat (LTR), human ubiquitin C promoter (hUBC), eGFP, a P2A sequence, the CD19-CAR and a 3′ LTR. The CD19-CAR contains an interleukin-2 signal peptide (IL-2 SP), the anti-CD19 FMC63 single chain variable fragment (scFv), a CD8 alpha hinge region, the transmembrane and intracellular domains of CD28, and the CD3-ζ intracellular signaling domain.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of immunology, medicine, organic chemistry, biochemistry, molecular biology, pharmacology, physiology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a rage of amino acids expressly specified in the claim. In certain embodiments, the disclosure contemplates that the “N-terminus of a peptide consists of an amino acid sequence,” which refers to the N-terminus of the peptide having the exact number of amino acids in the sequence and not more or having not more than a rage of amino acids specified in the claim however the C-terminus may be connected to additional amino acids, e.g., as part of a larger peptide. Similarly, the disclosure contemplates that the “C-terminus of a peptide consists of an amino acid sequence,” which refers to the C-terminus of the peptide having the exact number of amino acids in the sequence and not more or having not more than a rage of amino acids specified in the claim however the N-terminus may be connected to additional amino acids, e.g., as part of a larger peptide.

“Subject” refers any animal, preferably a human patient, livestock, or domestic pet.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the term “combination with” when used to describe administration with an additional treatment means that the agent may be administered prior to, together with, or after the additional treatment, or a combination thereof.

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free expression system. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a peptide disclosed herein in operable combination with a heterologous promoter.

Protein “expression systems” refer to in vivo and in vitro (cell free) systems. Systems for recombinant protein expression typically utilize somatic cells transfected with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids, and nucleotides. In the presence of an expression vectors, these extracts and components can synthesize proteins of interest. Cell-free systems typically do not contain proteases and enable labeling of the protein with modified amino acids. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

A “variant” refers to a chemically similar peptide sequence because of amino acid changes. In certain embodiments, a variant contains one or two, or more amino acid deletions or substitutions. In certain embodiments, the substitutions are conserved substitutions. In certain embodiments, a variant contains one, two, or ten or more, or ten or less amino acid additions. In certain embodiments, the additions may be to the N-terminus or the C-terminus. The variant may be substituted with one or more chemical substituents.

A conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. A variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted, or deleted without abolishing biological activity may be found using computer programs well known in the art. Variants can be tested in functional assays. Certain variants have less than 10%, and preferably less than 5%, and still more preferably less than 2% changes (whether substitutions, deletions, and so on). Variants can be prepared for testing by mutating a vector to produce appropriate codon alternatives for peptide translation.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q. The amino acid groups are also considered conserved substitutions.

As used herein, a “chimeric antigen receptor” or “CAR” refers to a protein receptor, which introduces an antigen specificity, via an antigen binding domain, onto cells to which it is expressed (for example T cells such as naive T cells, central memory T cells, effector memory T cells or combination thereof) thus combining the antigen binding properties of the antigen binding domain with the T cell activity (e.g. lytic capacity and self renewal) of T cells. A CAR typically includes an extracellular antigen-binding domain (ectodomain), a transmembrane domain and an intracellular signaling domain. The intracellular signaling domain generally contains at least one immunoreceptor tyrosine-based activation motif (ITAM) signaling domain, e.g. derived from CD3zeta, and optionally at least one costimulatory signaling domain, e.g. derived from CD28 or 4-1BB.

In order to improve the ability of immune cells to kill cancerous cells, T cells can be isolated from the blood of a patient and genetically altered to express chimeric antigen receptors (CARs) to specifically target proteins expressed on the surface of cancerous cells and stimulate an immune response. When put back into the patient, the cells attack the cancerous cells. Brentjens et al. report that T cells altered to bind CD19 can induce remissions of cancer in adults with chemotherapy-refractory acute lymphoblastic leukemia. Sci Transl Med, 2013, 5(177):177ra38.

Whole blood is composed of plasma, red blood cells (RBCs; or erythrocytes), platelets, and nucleated white blood cells, also referred to as leukocytes. The leukocytes can be further categorized into mononuclear cells and polymorphonuclear cells (or granulocytes). There are different techniques to obtain peripheral blood mononuclear cells (PBMCs), polymorphonuclear cells, leukocytes, or specific cell subsets, e.g., isolate specific cells directly by using flow cytometry, depleting red blood cells, centrifugation, and/or apheresis.

In a typical procedure, T cells are purified and isolated from blood or bone marrow. For example, T cells are collected via apheresis, a process that withdraws blood from the body and removes one or more blood components (such as plasma, platelets, or other white blood cells). The remaining blood is then returned back into the body. The cells are exposed to a recombinant vector, such as a lentiviral vector, that infects the cells in a way that a chimeric antigen receptor (CAR) protein is produced and presented in the cell membrane.

Before and/or after infecting the isolated cells with the recombinant vector, the cells may be induced to replicate. The genetically modified T cells may be expanded by growing cells in the laboratory until there are sufficient number of them. Optionally, these CAR T cells are frozen. The modified cells are then administered back to the patient. Various T cell subsets, as well as T cell progenitors and other immune cells such as natural killer (NK) cells, can be targeted with a CAR.

In certain embodiments, the targeting sequence in a chimeric antigen receptor refers to any variety of polypeptide sequences capable of selectively binding to a targeted molecule. The targeting sequences may be derived from variable binding regions of antibodies, single chain antibodies, and antibody mimetics. In certain embodiments, targeting sequence is a single-chain variable fragment (scFv) derived from an antibody. The targeting sequence is typically connected to intracellular domains by a hinge/transmembrane region, commonly derived from CD8 or IgG4. The intracellular domains may contain co-stimulatory domains such as CD80, CD86, 4-1BBL, OX40L and CD70 and/or CD28 linked to the cytoplasmic signaling domain of CD3zeta. See Sadelain et al. The basic principles of chimeric antigen receptor (CAR) design, Cancer Discov. 2013, 3(4): 388-398.

Peripheral blood mononuclear cells (PBMCs) may be isolated by leukapheresis. T cells can be enriched by mononuclear cells counter-flow elutriation and expanded by addition of anti-CD3/CD28 antibody coated paramagnetic beads for activation of T cells. Cells may be expanded, harvested, and cryopreserved in infusible medium sometime after the subject has had an allogeneic stem-cell transplantation.

Cells may be obtained by isolation from peripheral blood and optionally purified by fluorescent activated cells sorting e.g., mixing cells with fluorescent antibodies or other fluorescent agents (molecular beacons) and separating the cells by flow cytometry based fluorescent sorting. Another option for cells sorting is to provide magnetic particles that are conjugated to specific binding agents, such as antibodies against a particular antigen on a target cells surface. After mixing with a sample, the antibody bound cells are put through a purification column containing a matrix composed of ferromagnetic spheres. When placed on a magnetic separator, the spheres amplify the magnetic field. The unlabeled cells pass through while the magnetically labeled cells are retained within the column. The flow-through can be collected as the unlabeled cells fraction. After a short washing step, the column is removed from the separator, and the magnetically labeled cells are eluted from the column.

CD3 is expressed on T cells as it is associated with the T cells receptor (TCR). The majority of TCR are made up of alpha beta chains (alpha beta T-cells). Alpha beta T-cells typically become double-positive intermediates (CD4+CD8+) which mature into single-positive (CD4+CD8−) T helper cells or (CD4−CD8+) cytotoxic T cells. T helper cells interact with antigen presenting dendritic cells and B cells. Upon activation with cognate antigen by dendritic cells, antigen specific CD4 T cells can differentiate to become various types of effector CD4 T cells with specific roles in promoting immune responses.

T cells may be isolated and separated from a human sample (blood or PBMCs or bone marrow) based on the expression of alpha beta T cells receptor (TCR), gamma delta T cells receptor, CD2, CD3, CD4, CD8, CD4 and CD8, NK1.1, CD4 and CD25 and other combinations based on positive or negative selection. In certain embodiments, the immune cells are CD8+, CD4+, alpha beta T cells, delta gamma T cells, natural killer cells and/or double-negative alpha beta T cells.

In certain embodiments, methods comprise the steps of harvesting hematopoietic stem and progenitor cells from the peripheral blood or bone marrow of a subject or a doner. The subject or donor may be treated with one or more clinically approved hematopoietic stem and progenitor cell mobilization agents, for example, Granulocyte-Colony Stimulating Factor (G-CSF), to increase the number of hematopoietic stem and progenitor cells that can be collected by apheresis.

### Methods of Treating Cancer Using IL-37

In certain embodiments, this disclosure relates to therapeutics containing IL-37, chimeric antigen receptors, nucleic acids, and vectors encoding or containing the same. In certain embodiments, this disclosure relates to methods of treating cancer comprising administering interleukin-37, chimeric antigen receptors, a nucleic acid, or vector encoding the same to a subject in need thereof.

In certain embodiments, this disclosure relates to methods of treating cancer or a hematological malignancy comprising administering an effective amount of a recombinant IL-37 protein or a nucleic acid encoding IL-37 to a subject in need thereof. In certain embodiments, the subject is over 55 or 65 years of age.

In certain embodiments, the IL-37 protein or nucleic acid encoding IL-37 is administered during, after, or more than one day before administration of autologous or non-autologous T cells optionally comprising a chimeric antigen receptor. In certain embodiments, the T cells are contacted with to anti-CD3 and anti-CD28 antibodies providing activated T cells prior to administration.

In certain embodiments, this disclosure relates to methods of treating a hematological malignancy comprising administering an effective amount of T cells expressing a cancer targeting chimeric antigen receptor and administering a vector encoding a recombinant IL-37 protein to a subject in need thereof. In certain embodiments, the T cells are contacted with to anti-CD3 and anti-CD28 antibodies providing activated CAR T cells prior to administration.

In certain embodiments, this disclosure relates to methods of treating a hematological malignancy comprising administering an effective amount of T cells expressing a cancer targeting chimeric antigen receptor and expressing a recombinant IL-37 protein to a subject in need thereof. In certain embodiments, the subject is over 55 or 65 years of age. In certain embodiments, the cancer targeting chimeric antigen receptor and the recombinant IL-37 protein are expressed in a single vector. In certain embodiments, the cancer targeting chimeric antigen receptor and the recombinant IL-37 protein are expressed in separate vectors.

In certain embodiments, this disclosure relates to methods of treating a hematological malignancy comprising administering an effective amount of T cells expressing a cancer targeting chimeric antigen receptor in combination with a recombinant IL-37 protein or a nucleic acid encoding IL-37 to a subject in need thereof. In certain embodiments, the subject is over 55 or 65 years of age.

In certain embodiments, the methods reported herein are done in combination with administering another anticancer agent to the subject. In certain embodiments, the nucleic acid encoding IL-37 is administered in combination with another anticancer agent. In certain embodiments, administrating T cells expressing a nucleic acid encoding IL-37 is administered in combination with another anticancer agent. In certain embodiments, the T cell are expressing a chimeric antigen receptor. In certain embodiments, the anticancer agent is a checkpoint inhibitor, an anti-PD-1, anti-PD-L1 anti-CTLA4 antibody or combinations thereof. In certain embodiments, the anti-CTLA4 antibody is ipilimumab or tremelimumab. In certain embodiments, the anti-PD1 antibody is nivolumab, pembrolizumab, or cemiplimab. In certain embodiments, the anti-PD-L1 antibody is atezolizumab, avelumab, or durvalumab.

In certain embodiments, the subject to be treated has or is diagnosed with a hematological malignancy and has previously received a bone marrow or hematopoietic stem cell transplant, e.g., wherein stem cells are collected from blood or bone marrow.

In certain embodiments, the nucleic acid encoding IL-37 or the recombinant IL-37 protein or T cells optionally expressing a chimeric antigen receptor are administered to a subject with a lymphodepleted environment due to prior or concurrent administration of a lymphodepleting agent. In certain embodiments, the of lymphodepleting agent is cyclophosphamide, fludarabine, or combination thereof.

In certain embodiments, the hematological malignancy is selected from leukemia, acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), chronic myeloid leukemia (CML), myeloproliferative neoplasms (MPNs), and lymphomas, Hodgkin's lymphomas, and non-Hodgkin's lymphomas such as Burkitt lymphoma, B-cell lymphoma.

In certain embodiments, this disclosure relates to vectors encoding a chimeric antigen receptor and IL-37. In certain embodiments, the chimeric antigen receptor and IL-37 are separated by a self-cleaving spacer, e.g., GSGATNFSLLKQAGDVEENPGP (SEQ ID NO: 21). In certain embodiments, IL-37 is connected to the N-terminal of the self-cleaving spacer and the chimeric antigen receptor is connected to the C-terminal of the self-cleaving spacer, and IL-37 has the amino acid sequence of MSFVGENSGVKMGSEDWEKDEPQCCLEDPAGSPLEPGPSLPTMNFVHTSPKVKN LNPKKFSIHDQDHKVLVLDSGNLIAVPDKNYIRPEIFFALASSLSSASAEKGSPILLGVSK GEFCLYCDKDKGQSHPSLQLKKEKLMKLAAQKESARRPFIFYRAQVGSWNMLESAAHP GWFICTSCNCNEPVGVTDKFENRKHIEFSFQPVCKAEMSPSEVSD (SEQ ID NO: 18) or variants thereof, e.g., have greater than 60, 70, 80, 85, 90, 95, 98 or 99% sequence identity.

In certain embodiments, this disclosure relates to methods of treating cancer or a hematological malignancy comprising administering an effective amount of a nucleic acid encoding IL-37 to a subject in need thereof. In certain embodiments, the subject is over 55 or 65 years of age.

In certain embodiments, the nucleic acid encoding IL-37 is administered during, after, and/or more than one day before administration of autologous or non-autologous T cells optionally comprising a chimeric antigen receptor.

In certain embodiments, this disclosure relates to methods of treating a hematological malignancy comprising administering an effective amount of T cells expressing a cancer targeting chimeric antigen receptor and administering a vector encoding a recombinant IL-37 protein to a subject in need thereof.

In certain embodiments, this disclosure relates to methods of treating a hematological malignancy comprising administering an effective amount of T cells expressing a cancer targeting chimeric antigen receptor and expressing a recombinant IL-37 protein to a subject in need thereof. In certain embodiments, the subject is over 55 or 65 years of age. In certain embodiments, the cancer targeting chimeric antigen receptor and the recombinant IL-37 protein are expressed in a single vector. In certain embodiments, the cancer targeting chimeric antigen receptor and the recombinant IL-37 protein are expressed in separate vectors.

In certain embodiments, this disclosure relates to methods of treating a hematological malignancy comprising administering an effective amount of T cells expressing a cancer targeting chimeric antigen receptor in combination with administering a nucleic acid encoding IL-37 or a recombinant IL-37 protein to a subject in need thereof. In certain embodiments, the subject is over 55 or 65 years of age.

In certain embodiments, the nucleic acid encoding IL-37 is administered in combination with another anticancer agent. In certain embodiments, the anticancer agent is a checkpoint inhibitor, an anti-PD-1, anti-PD-L1 anti-CTLA4 antibody or combinations thereof. In certain embodiments, the anti-CTLA4 antibody is ipilimumab or tremelimumab. In certain embodiments, the anti-PD1 antibody is nivolumab, pembrolizumab, or cemiplimab. In certain embodiments, the anti-PD-L1 antibody is atezolizumab, avelumab, or durvalumab.

In certain embodiments, this disclosure relates to methods of treating cancer comprising contacting isolated T-cells with interleukin-37 providing interleukin-37 activated T cells and administering and effective amount of interleukin-37 activated T cells to a subject in need thereof. In certain embodiments, the interleukin-37 activated T cells increase gene expression levels of Pdcd1, Lat, Stat4, or combinations thereof. In certain embodiments, the interleukin-37 activated T cells are CD4 positive T cells and increase gene expression levels of Pdcd1, Lat, and Stat4. In certain embodiments, the interleukin-37 activated T cells are CD8 positive T cells and increase gene expression levels of Lat. In certain embodiments, the T cells comprise a nucleic acid or vector encoding a chimeric antigen receptor.

In certain embodiments, this disclosure relates to implementing methods disclosed herein wherein the subject have a compromised immune system is over 55 or 65 years old.

In certain embodiments, the subject is taking immunosuppressive medications.

In certain embodiments, the subject is being treated with chemotherapy agents for solid tumor or hematologic malignancies

In certain embodiments, the subject is a recipient of chimeric antigen receptor (CAR)-T-cell therapy or hematopoietic stem cell transplant (within 2 years of transplantation or taking immunosuppression therapy).

In certain embodiments, the subject is a recipient of solid-organ transplant and taking immunosuppressive therapy.

In certain embodiments, the subject is diagnosed with primary immunodeficiency (e.g., DiGeorge syndrome, Wiskott-Aldrich syndrome).

In certain embodiments, the subject is being treated with high-dose corticosteroids (i.e., 10 mg or more of prednisone or equivalent per day when administered for greater than 2 weeks), alkylating agents, antimetabolites, transplant-related immunosuppressive drugs, cancer chemotherapeutic agents classified as immunosuppressive, tumor-necrosis (TNF) blockers, and other biologic agents that are immunosuppressive or immunomodulatory (e.g., B-cell depleting agents).

In certain embodiments, methods performed herein are done in combination with administering another anticancer agent. In certain embodiments, the anticancer agent is abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

In certain embodiments, the anticancer agent is an anti-PD-1, anti-PD-L1 anti-CTLA4 antibody or combinations thereof, such as an anti-CTLA4 (e.g., ipilimumab, tremelimumab) and anti-PD1 (e.g., nivolumab, pembrolizumab, cemiplimab) and anti-PD-L1 (e.g., atezolizumab, avelumab, durvalumab).

### Pharmaceutical Compositions

In certain embodiments, this disclosure contemplates pharmaceutical compositions comprising peptides disclosed herein, or nucleic acids and vectors encoding the same, and optionally at least one pharmaceutically acceptable carrier, diluent, or excipient. The pharmaceutical compositions can be administered in any suitable manner that allows the composition to enter the circulation, such as intravenously, via injection or infusion, or in any other suitable manner (including oral administration, subcutaneous administration, intramuscular administration, administration through the skin, intranasal administration, administration via the lungs, etc.).

The term “pharmaceutical formulation” refers to a preparation which is in such form as to permit the biological activity of the active ingredient (the polypeptide, nucleic acid, vector) to be effective, and which contains no additional components which are unacceptably toxic to a subject to which the formulation would be administered. Such formulations are sterile, “Pharmaceutically acceptable” excipients (vehicles, additives) are those which can reasonably be administered to a subject mammal to provide an effective dose of the active ingredient employed.

The term “excipient” as used herein refers to an inert substance which is commonly used as a diluent, vehicle, preservative, surfactant, binder, carrier, or stabilizing agent for compounds which impart a beneficial physical property to a formulation. The skilled person is familiar with excipients suitable for pharmaceutical purposes, which may have particular functions in the formulation, such as stabilization, preservation, etc.

A “sterile” formulation is aseptic or free or essentially free from all living microorganisms and their spores. This is readily accomplished by filtration through sterile filtration membranes.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. The formulation comprises an aqueous carrier. The aqueous carrier is in particular a buffer.

As used herein, “buffer” refers to a buffered solution that resists changes in pH by the action of its acid-base conjugate components. The pH of the formulation is typically in the range 5.0 to 7.5, wherein each value is understood to encompass a range of plus or minus 0.2. The most advantageous pH will depend on the buffer comprised in the formulation. Hence, a formulation comprising a phosphate buffer, which preferably has a pH in the range of 6.5 to 7.5, preferably 6.9, 7.0, 7.1, e.g. 7.1.

In certain embodiments, formulations comprise the active ingredient at a concentration that is suitable for clinical purposes, which includes concentrations used in stock solutions for dilution prior to use on the patient. Typical concentrations comprise the non-limiting examples of concentrations in the range of 0.1 to 150 mg/mL, such as 1-100 mg/mL, 5-80 mg/mL, or 10-40 mg/mL, preferably 10 mg/mL, wherein each value is understood to optionally encompass a range (e.g. a value of 10 optionally encompasses a range of 8 to 12 mg/mL).

The formulation may further comprise stabilizing agents, such as a polyols. A “polyol” is a substance with multiple hydroxyl groups, and includes sugars (reducing and nonreducing sugars), sugar alcohols and sugar acids, A polyol may optionally be included in the formulation, for instance to improve stability. In certain embodiments, polyols herein have a molecular weight which is less than about 600 kD (e.g. in the range from about 120 to about 400 kD). A “reducing sugar” is one which contains a hemi-acetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, and raffinose. Mannitol, xylitol, erythritol, threitol, sorbitol and glycerol are examples of sugar alcohols. As to sugar acids, these include L-gluconate and metallic salts thereof. Where it desired that the formulation is freeze-thaw stable, the polyol is preferably one which does not crystallize at freezing temperatures (e.g. −20 degrees C.) such that it destabilizes the peptide in the formulation. In certain embodiments, nonreducing sugars such as sucrose and trehalose are examples of polyols, with sucrose being preferred, despite the solution stability of trehalose.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, hydrophilic polymers such as polyvinyl pyrrolidone, cellulose based substances, polyethylene glycol, sodium carboxymethylcellulose, polyacrylates, waxes, gelatin, polyethylene polyoxypropylene block polymers, polyethylene glycol and antioxidants including ascorbic acid and methionine; preservatives; low molecular weight (less than about 10 residues) polypeptides; proteins; and amino acids such as glycine, glutamine, asparagine, histidine, arginine, or lysine. In advantageous embodiments, the excipient may be one or more selected from the list consisting of NaCl, trehalose, sucrose, mannitol, or glycine.

The disclosure also encompasses products obtainable by further processing of a liquid formulation, such as a frozen, lyophilized or spray-dried product. Upon reconstitution, these solid products can become liquid formulations as described herein (but are not limited thereto). In its broadest sense, therefore, the term “formulation” encompasses both liquid and solid formulations. However, solid formulations are understood as derivable from the liquid formulations (e.g. by freezing, freeze-drying or spray-drying), and hence have various characteristics that are defined by the features specified for liquid formulations herein.

In certain embodiments, the formulations are isotonic in relation to human blood. Isotonic solutions possess the same osmotic pressure as blood plasma, and so can be intravenously infused into a subject without changing the osmotic pressure of the subject's blood plasma.

### Kits

In certain embodiments, this disclosure relates to kits containing materials useful for the treatment of a cancer as described above is provided. In certain embodiments, the kit comprises a container, a product label and a package insert. Suitable containers include, for example, bottles, vials, syringes, etc. The containers may be of a variety of materials such as glass or plastic. The container holds the composition which is effective in treating the condition and may have a sterile access port (for example the container may be an intravenous solution bag or a vial having a stopper pierceable by a hypodermic injection needle). At least one active agent in the composition is peptide, nucleic acid, or vector as disclosed herein. The product label on, or associated with, the container indicates that the composition is used for treating the condition of choice. In certain embodiments, the kit may further comprise a second container comprising a pharmaceutically acceptable buffer, such as a phosphate buffer saline or a citrate buffered saline as described herein. It may further include other materials desirable from a user or commercial standpoint, including other buffers, diluents, filters, needles, and syringes. In certain embodiments, a dosage unit form can be e.g. in the format of a prefilled syringe, an ampoule, cartridge or a vial.

In certain embodiments, this disclosure relates to kits or articles of manufacture, comprising a polypeptide or the formulation thereof as disclosed herein and instructions for use by, e.g., a healthcare professional. The kits or articles of manufacture may include a vial or a syringe containing the formulation as described herein.

Preferably, the vial or syringe is composed of glass, plastic, or a polymeric material chosen from a cyclic olefin polymer or copolymer. The syringe, ampoule, cartridge, or vial can be manufactured of any suitable material, such as glass or plastic and may include rubber materials, such as rubber stoppers for vials and rubber plungers and rubber seals for syringes and cartridges.

In certain embodiments, the kit may further comprise instructions for use and/or a clinical package leaflet. In any embodiment of the products as defined herein, this disclosure also encompasses the presence of packaging material, instructions for use, and/or clinical package leaflets, e.g., as required by regulatory aspects.

### Interleukin-37 Improves T-Cell-Mediated Immunity and Chimeric Antigen Receptor T-Cell Therapy in Aged Backgrounds

Experiments were performed to determine whether IL-37 impacts the function of aged endogenous and CAR T-cells. Data indicates that transgenic expression of IL-37 in aged mice and treating aged mice with recombinant human IL-37 (rIL-37) improves the function of non-engineered and CAR T-cells. Treating aged mice with rIL-37 restores the expression of key genes involved in T-cell activation which decline with normal aging and reduces the surface expression of multiple immunoinhibitory proteins on aged CD4+ and CD8+ T-cells to youthful levels. IL-37 signaling directly opposes TNF-α signaling and downregulates PD-1 surface expression on aged T-cells. Additionally, rIL-37 treatment of aged mice augments cytokine production by endogenous T-cells, and when combined with CAR T-cell therapy, improves their therapeutic capacity in a murine model of B-ALL. Because the expression of the IL-37 gene decreases in an age-dependent manner in human monocytes, methods of increasing circulating IL-37 levels in aged backgrounds may represent a strategy to overcome aging-associated T-cell senescence.

Transgenic IL-37 expression in aged mice rejuvenated the function of aged B-progenitor cells and abrogated the selection of B-cells harboring oncogenic mutations; thereby, preventing leukemogenesis. Experiments reported herein, indicate that IL-37 impacted the function of mature immune cells. The IL-37 gene expression levels were significantly lower in monocytes isolated from donors 65 years of age or older relative to their younger counterparts, suggesting that inflammaging is accompanied by lower levels of IL-37 production from innate immune cells.

Treating aged mice with recombinant IL-37 abrogates aging-associated splenomegaly. This change was accompanied by restoring a youthful balance of CD4+ to CD8+ T-cells and evoking youthful gene expression programs in T-lymphocytes. Of particular interest, the gene expression levels of the linker for the activation of T-cells (Lat) was found to be increased to youthful levels in aged CD4+ and CD8+ T-cells recovered from old mice receiving rIL-37 treatment. This observation suggests that rIL-37 treatment augments TCR-mediated signaling in aged T-cells. Indeed, T-cells isolated from aged mice receiving rIL-37 responded more robustly to αCD3/αCD28 stimulation which mimics peptide-MHC/TCR activation. Indeed, both T-helper cells and cytotoxic lymphocytes exhibited significantly enhanced IL-2 and IFN-γ production with this mode of stimulation. Recombinant IL37 treatment of aged mice also significantly reduced Pdcd1 (the gene encoding for PD-1) and significantly increased Stat4 gene expression levels in T-helper cells, suggesting an attenuation of T-cell exhaustion and enhanced IL-12-mediated signaling (which may play a role in augmenting IFN-γ production from aged T-cells).

In addition to modifying gene expression profiles, rIL-37 treatment of aged mice resulted in decreased surface expression of the immunosuppressive molecules PD-1, Tim-3, and TIGIT on activated T-cells coincident with increased proliferation after in vitro stimulation. The increase in the proliferation in aged T-cell is notable because aged microenvironments are capable of potently suppressing the proliferation of young and aged T-cells. Given that aging-associated T-cell dysfunction has been attributed to increased levels of PD-1, Tim-3, and TIGIT, experimental results suggest that IL-37-mediated rejuvenation of aged T-cells is partially attributed to its ability to downregulate the surface expression of these immunoinhibitory proteins on T-lymphocytes. In all, these results demonstrate that IL-37 treatment reprograms gene expression profiles in aged T-cells resulting in more robust effector functions and an increased threshold for T-cell exhaustion post-stimulation.

The ability of IL-37 to restore youthful gene expression profiles, mitigate immunosuppressive mechanisms, and enhance effector T-cell function is attributed to both direct effects on T-cells and modulation of the immune environment. Transgenic expression of IL-37 and rIL-37 treatment attenuated aging-associated increases in circulating IL-1β, IL-6, and TNF-α levels. Given that chronic TNF-α exposure suppresses T-cell receptor signaling, blocking TNF-α enhances CD8+ T-cell responses in murine models of melanoma, and TNF-α/PD-1 gene expression levels are positively correlated in patients with melanoma, experiments were performed to determine whether IL-37 antagonized TNF-α signaling in aged T-cells. In immune cells, TNF-alpha stimulation potently activates NF-κB, which has multiple binding sites in the T-cell PD-1 promoter region. The experiments indicate rIL-37 directly antagonized TNF-α-mediated NF-κB activation. Furthermore, IL-37 treatment significantly reduced PD-1 surface expression and genes activated downstream of both PD1 and TNF-α signaling pathways (TMEM16F, GM130, PD-1, and SHP2). Directly stimulating aged T-cells with rIL-37 also augmented the expression levels of genes which promote interferon production (IFN-γ, TBK1, and IRF3), coincident with increased IFN-γ production from aged T-cells after αCD3/αCD28 stimulation.

In addition to IL-37-mediated cell autonomous changes in aged T-cells, aging-associated increases in myelopoiesis were abrogated after treating aged mice with rIL-37. Similarly, splenic DC and macrophage populations were also decreased to youthful levels after aged mice received rIL-37 treatment. The reduction in myeloid cells in the bone marrow and spleens of aged mice treated with rIL-37 likely contributed to the significantly lower levels of circulating pro-inflammatory cytokines and more robust T-cell effector functions.

IL-37 binds the IL-18Ra and IL-1R8 receptors, which are expressed on myeloid cells and T-cells, and attenuates the production of pro-inflammatory cytokines by inhibiting transforming growth-factor-β-activated protein kinase 1 (TAK1), NF-κB, and MAPK activity. The “renormalization” of the inflammatory microenvironment in aged mice is consistent with reported protective effects of IL-37 in other pathological inflammatory settings including endotoxin shock syndrome, lung and spinal cord injury, colitis, coronary artery disease, and arthritis.

Aging in mice and humans is associated with extensive immunological changes including the onset of chronic inflammation and the development of compromised T-cell-mediated immunity. Augmented immunosuppressive mechanisms in aged individuals are postulated to contribute to higher cancer incidence. In addition to elevated cancer incidence, cancer-related mortality rates are significantly higher in older patients. The failure to achieve similar survival outcomes in younger and older patients with cancer has been partially attributed to the inability to achieve effective chemotherapy dosages in older patients due to toxicity complications. Given that chemotherapies are less effective in older patients, other therapeutic options, such as treatments using immunotherapies, are beginning to be used to treat older patients with solid and hematological malignancies.

Indeed, CAR T-cell therapy is currently being used to treat relapsed and refractory B-ALL and DLBCL with new clinical trials open to test the efficacy of this cell-based therapy as a frontline option. Despite the success of CAR T-cell therapy, between 20 and 50 percent of the pediatric and adult patients receiving this form of immunotherapy will relapse within 2 years of treatment. The failure to achieve durable responses in patients receiving CAR T-cell therapy has resulted from receiving low potency CAR T-cells and the loss of target antigens on cancer cells. The incidence of most leukemias rises dramatically in individuals over 65, and mortality rates are higher in geriatric patients. Given the impact of the immune microenvironment on cancer progression and the immunological decline associated with aging, there is a growing need to implement immunotherapies in aged recipients.

Experiments reported herein indicated functional defects in aged endogenous T-cells are transferable to engineered T-cells and are not completely overcome by the introduction of a CAR. Aging-associated increases in chronic inflammation, the onset of splenomegaly, and the accumulation of myeloid populations in the bone marrow and spleen can be prevented by the anti-inflammatory cytokine IL-37. Importantly, treating aged mice with rIL-37 reduces TNF-α signaling and significantly decreased the surface expression of PD-1 on naïve CD4+ and CD8+ T-cells. This effect was not limited to endogenous T-cells, as demonstrated by the results that rIL-37 treatment also prevented high PD-1 surface expression on aged CAR T-cells. Impressively, the function of endogenous and CAR T-cells was improved by rIL-37 treatment, leading to increased cytokine production ex vivo and the augmented protection of mice with B-ALL. Recombinant IL-37 treatment boosts T-cell-mediated immunity in aged backgrounds and increases the efficacy of aged CAR T-cells.

### Interleukin-37 Suppresses Inflammaging, and Decreased Levels are Observed in Aged Human Monocytes

Transgenic expression of the anti-inflammatory cytokine IL-37 improves hematopoiesis and the function of B-progenitor cells in aged mice, which is driven by reducing aging-associated inflammation. Experiments were performed to determine whether treating aged mice (≥24 months old) with rIL-37 impacts systemic inflammation relative to levels observed in IL-37 transgenic (IL-37 Tg) mice. Aged (24 months old) wild-type mice were treated with control immunoglobulin (Control Ig) or rIL-37 every 2 days for 2 weeks. This rIL-37 treatment significantly decreased circulating tumor necrosis factor-alpha (TNF-α), interleukin-1 beta (IL1β), and interleukin-6 (IL-6) levels in aged mice which were comparable to observations in aged IL-37tg mice. Given the ability of IL-37 to mitigate inflammaging in aged mice, experiments where designed to determine whether IL-37 levels declined in humans with age. The R2 database was evaluated for studies where TL-37 gene expression profiles were available for healthy donors. A repository was analyzed. The age range of donors in the database was 15-55 years old, reflective of young to middle-aged healthy humans. An arbitrarily cutoff was set the for young donors as those between 15 and 39 years of age and middle-aged as donors between 40-55 years of age. When the data were binned into these groups, A slight decrease in IL-37 gene expression levels was found in leukocytes recovered from middle-aged (n=7) relative to young donors (n=30).

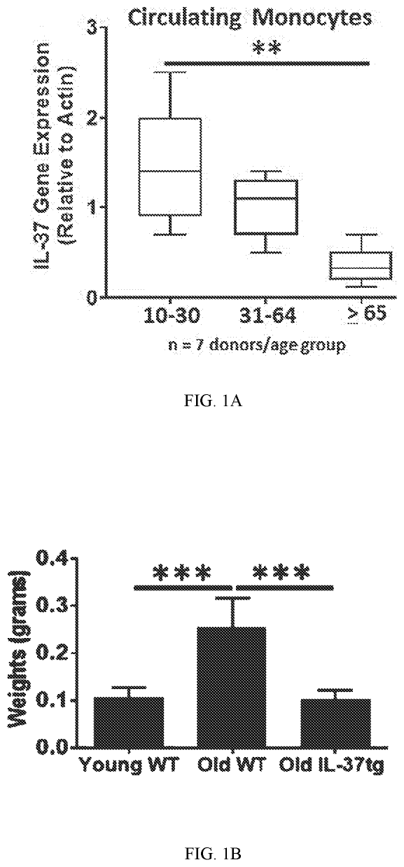

To assess the impact of advanced age on IL37 expression levels, peripheral blood mononuclear cells (PBMCs) were obtained from healthy donors of various ages, including those over 65 years of age. Monocytes, which are major producers of IL-37, were purified from PBMCs, and IL-37 and actin gene expression levels were compared. A decreased trend in IL-37 gene expression levels was found in monocytes isolated from donors between 10 and 30 and those 31-64 years of age (FIG. 1). In donors 65 and older, IL-37 gene expression levels in monocytes were significantly lower than those observed in monocytes isolated from donors between 10 and 30 years of age. This data indicates that IL-37 may suppress aging-associated chronic inflammation and that reduced IL-37 levels in aged monocytes may contribute to the onset of inflammaging in humans.

### Interleukin-37 Abrogates Splenomegaly and Restores a Youthful T-Cell Distribution in Aged Mice

IL-37 transgenic (IL-37 Tg) mice have improved B-progenitor cell function. Experiments were performed to determine whether recombinant IL-37 (rIL-37) treatment of aged mice mitigated aging-associated changes in hematopoiesis. Aged (24 months old) wild-type mice were treated with control immunoglobulin (Control Ig) or rIL-37 every 2 days for two weeks. rIL-37 treatment in aged mice prevented the aging-associated accumulation of myeloid progenitor cells in the bone marrow and macrophages in the spleen. Despite altering the relative distribution of myeloid cells, rIL-37 treatment did not change the absolute number of hematopoietic stem cells, B-progenitor cells, splenic-derived B-cells, or splenic-derived T-cells in aged mice. Aging is associated with extensive microarchitectural changes in the spleen including the onset of splenomegaly as a result of prolonged stimulation mediated by chronic inflammation or neoplastic lesions. In addition to abrogating aging-associated chronic inflammation, transgenic expression of IL-37 also significantly reduced splenomegaly in aged mice (FIG. 1B). Similar to its impact on hematopoiesis, treating aged mice with rIL-37 also mitigated splenomegaly.

Experiments were performed to determine how rIL-37 treatment of aged, naïve mice impact the distribution of splenic-derived immune cells and their basal activation state. Young (2 months old) and aged (24 months old) mice were treated with control immunoglobin (Control Ig) or rIL-37 using the protocol described above. In these experiments, similar percentages of splenic-derived CD4+ T-cells were found in young, naïve mice treated with Control Ig and rIL-37. In aged mice treated with Control Ig, we observed a slight decrease in the percentage of splenic-derived CD4+ T-cells relative to all treatment groups, whereas rIL-37 treatment led to a noticeable, although not statistically significant (p=0.059), increase in the representation of T-helper cells. Similarly, equivalent percentages of splenic-derived CD8+ T-cells were observed in young, naive mice treated with Control Ig and those treated with rIL-37. In contrast to the slight decrease in the representation of CD4+ T-cells observed in aged mice treated with Control Ig, the percentage of CD8+ T-cells was noticeably, yet insignificantly (p=0.084), higher than those observed in all treatment groups. Interestingly, the trend toward increased representation of splenic-derived CD8+ T-cells in aged mice was mitigated by rIL-37 treatment. Overall, these data demonstrate that treating aged mice with rIL-37 abrogates aging-associated splenomegaly and restores the representation of CD4+ and CD8+ T-cells to youthful levels (FIG. 1C), whereas treating young mice with this anti-inflammatory cytokine does not impact the distribution of splenic-derived T-cells.

### IL-37 Promotes Youthful Gene Expression Profiles in Aged T-Cells and Reduces the Surface Expression of Immunoinhibitory Proteins

Given the ability of rIL-37 treatment to restore a youthful CD4+ to CD8+ T-cell distribution in aged mice, how treatment with this anti-inflammatory cytokine impacted the gene and surface expression of regulators of T-cell activation was determined. After 2 weeks of treatment, CD4+ T-cells isolated from aged mice treated with rIL-37 exhibited gene expression profiles that phenocopied CD4+ T-cells isolated from young mice. When aged mice were treated with Control Ig, CD4+ T-cells exhibited a trend toward higher gene expression levels of Pdcd1 (the gene encoding programmed cell death protein 1 [PD-1]) and significantly lower levels of Lat and Stat4. Treatment of aged mice with rIL-37 reversed these phenotypes in CD4+ T-cells to youthful levels, which was comparable to young mice treated with Control Ig and rIL-37. Furthermore, a significant increase in Prf1 (the gene encoding perforin) expression levels were observed in CD4+ T-cells isolated from aged mice treated with rIL-37. Unlike rIL-37-mediated gene expression alterations in aged CD4+ T-cells, treating young mice with rIL-37 did not alter Cd3e, Cd28, Prf1, Pdcd1, Lat, Il12rb1, or Stat4 gene expression levels in CD4+ T-cells. In addition to altering gene expression profiles in aged CD4+ T-cells, treating aged mice with rIL-37 also decreased the surface expression of the immunoinhibitory proteins Tim-3 and TIGIT on aged T-cells, whereas CD28 surface levels remained unchanged. Despite the aging-associated increase in Pdcd1 gene expression levels in aged CD4+ T-cells, PD-1 surface expression on naïve CD4+ T-cells was negligible and not impacted by rIL-37 treatment, which is consistent with PD-1 expression being induced on activated T-cells.

Unlike, changes observed in CD4+ T-cells in aged mice receiving rIL-37 treatment, aging-associated gene expression changes in CD8+ T-cells were largely unchanged with rIL-37 treatment with the exception of restoring youthful levels of Lat. Similar to CD4+ T-cells, treating aged mice with rIL-37 also significantly decreased TIGIT surface levels on naïve CD8+ T-cells, whereas Tim3 and CD28 surface expression was not impacted by rIL-37 treatment in young or aged mice.

In addition to assessing the impact of rIL-37 treatment on aged T-lymphocytes, its impact on aged myeloid cells was also determined. Treating aged mice with rIL-37 also led to a reduction (albeit insignificant) in splenic dendritic cells and macrophages consistent with an abrogation of splenomegaly (FIG. 1). Despite decreasing the percentages of splenic-derived dendritic cells, which are the principal activators of naïve T-cells (Henry et al., 2008, 2010), rIL-37 treated did not rejuvenate their upregulation of the costimulatory molecules CD40, CD80, and CD86 to youthful levels after ex vivo stimulation with LPS.

These data demonstrate that treating aged mice with rIL-37 alters the activation threshold of naïve CD4+ and CD8+ T-cells, by increasing the expression of genes involved in T-cell activation (Stat4 and Lat) and decreasing the surface expression of immunoinhibitory proteins (Tim-3 and TIGIT).

Recombinant IL-37 Treatment Improves T-Cell Function in Aged Mice Given that IL-37 treatment rejuvenated gene expression profiles and suppressed the surface

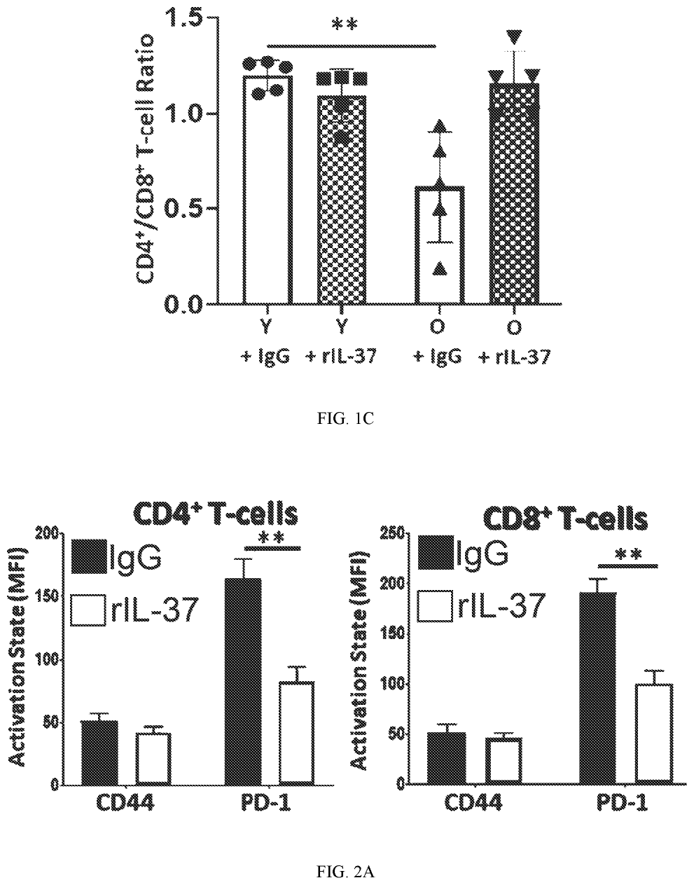

expression of immunoinhibitory proteins, the impacted of T-cell function was assessed. CD4+ and CD8+ T-cells were purified from aged (24 months old) wild-type mice treated every other day for 2 weeks with Control Ig or rIL-37 and stimulated in vitro with αCD3/αCD28 for 3 days. rIL-37 treatment significantly mitigated T-cell exhaustion indicative of similar T-cell expansion observed between T-cell isolated from aged mice treated with rIL-37 and young mice treated with Control Ig or rIL-37. In contrast, T-cell proliferative defects were observed in aged T-cell isolated from aged mice treated with Control Ig, where significant difference were apparent by Day 2 of culture and became more pronounced by day 4 post-stimulation. Furthermore, treating aged mice with rIL-37 significantly reduced the surface expression of PD-1 on effector CD4+ and CD8+ T-cells, whereas CD44 surface levels remain unchanged. T-cells stimulated from aged mice treated with rIL-37 were more functional than T-cells activated from aged mice treated with Control Ig (FIG. 2A). Significant increases in interleukin-2 (IL-2) and interferon-gamma (IFN-γ) production were observed at the population (percentage) and per cell (mean fluorescence intensity) levels when T-cells were stimulated ex vivo from rIL-37-treated but not Control Ig-treated aged mice. In summary, these data demonstrate that treating aged mice with recombinant IL-37 effectively improves T-cell responses.

Pro-inflammatory cytokines, such as TNF-α, are potent inducers of PD-1/PD-L1 surface expression on immune cells. Given that treating aged mice with rIL-37 significantly reduced chronic inflammation and was particularly effective at lowering circulating TNF-α levels, experiments were performed to determine if rIL-37 directly counteracted TNF-α signaling and its ability to induce PD-1 surface expression on aged T-cells. In immune cells, TNF-α is a potent inducer of NF-κB activation and NF-κB binding sites are located in the PD-1 promoter. Treating aged CD4+ and CD8+ T-cells with recombinant TNFα (rTNF-α) significantly augmented NF-κB activation in T-cells which correlated with increased PD-1 surface expression on effector T-cells.

Experiments were performed to determine whether rIL-37 stimulation could reduce NF-κB activation in TNF-α stimulated aged T-cells. rIL-37 abrogated the TNF-α induced NF-κB activation in aged T-cells and significantly decreased PD-1 surface expression.

To determine whether IL-37 altered T-cell homeostasis prior to stimulation, gene expression profiling of targets induced (TMEM16F, GM130, PD-1, and SHP2) and suppressed (IFN-γ, TBK1, and IRF3) by TNF-α and PD-1 signaling was performed in aged naïve T-cells treated with Control Ig or rIL-37. In young naïve T-cells, low basal expression of genes that are induced and suppressed by TNF-α and PD-1 signaling were observed. Furthermore, rIL-37 treatment did not impact the expression of these genes in young naïve T-cells.

In contrast, aged naïve T-cells exhibited high gene expression levels of TMEM16F, GM130, PD1, and SHP2 suggesting that these programs are primed for induction in aged T-cells. Furthermore, treating aged naïve T-cells with rIL-37 significantly increased the homeostatic expression of genes suppressed by TNF-α and PD-1 signaling, particularly those involved in interferon production. Taken together, these data demonstrate that rIL-37 improves the function of aged T-cells which is mediated, in part, by the ability of rIL37 treatment to directly oppose TNF-α-induced programs in aged T-cells.

Recombinant IL-37 Treatment Protects Aged Mice from B-ALL Pathogenesis in a T-Cell Dependent Manner

Two hallmarks of aging are the onset of chronic inflammation and compromised immunity, which are postulated to contribute to numerous aging-associated pathologies including cancer. Transgenic expression of IL-37 improves hematopoiesis and the function of B-progenitor cells in aged mice driven by reducing aging-associated inflammation. Experiments were performed to determine if reducing aging-associated chronic inflammation impacts leukemia development. Aged wild-type and IL-37 transgenic (IL-37tg) mice were transplanted with BCR-ABL1+/Arf-null B-ALL cells. Due to the presence of a strong driver mutation (BCR-ABL1) and the lack of a potent tumor suppressor (Arf), these cells are capable of establishing leukemia in mice without myeloablation, which leaves the immune system unperturbed. After transplantation into aged wild-type mice, all mice succumbed to disease within 2 months post-injection of B-ALL cells. The transgenic expression of IL-37 in aged mice resulted in a significant extension of survival, such that almost half of the mice injected with B-ALL cells survived for over 2 months demonstrating that IL37 expression in aged mice protects against B-ALL progression.

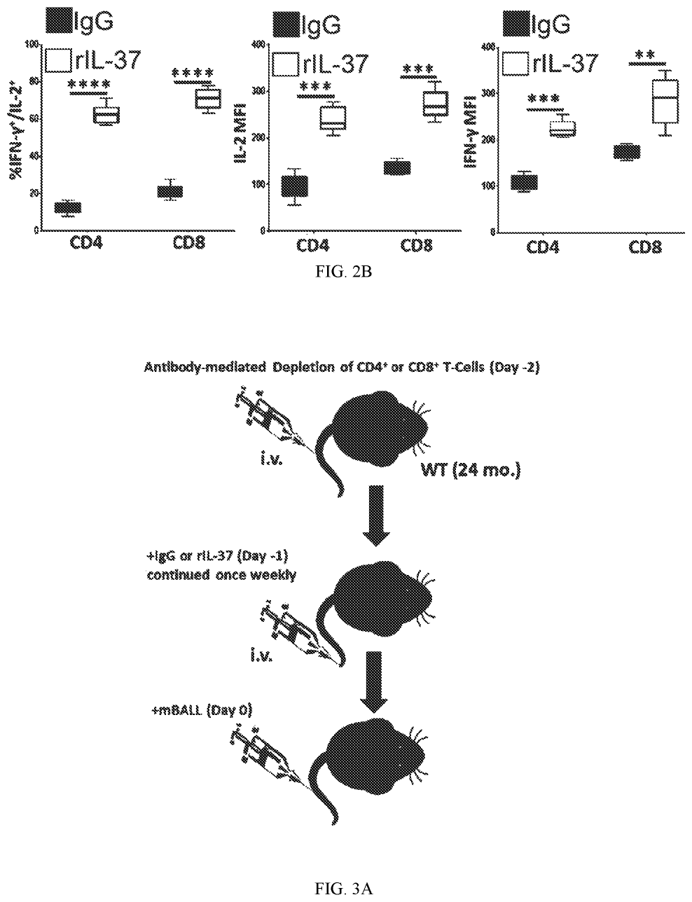

Experiments were performed to determine whether treating aged mice with rIL-37 could improve T-cell-mediated anti-leukemia responses. To this end, aged mice were treated with Control Ig or T-cell depleting antibodies followed by treatment with Control Ig or rIL-37 prior to injection with BCR-ABL1+/Arf-null B-ALL cells (FIG. 3A). Mice were treated with control Ig or rIL-37 for the duration of this experiment.

Given the aggressive nature of this leukemia, all mice succumbed to disease within 42 days post-injection if left untreated (FIG. 3B). Impressively, 60% of mice treated continuously with rIL-37 exhibited survival for greater than 3 months post-injection of B-ALL cells (FIG. 3B). This protective effect was abrogated when CD4+ and CD8+ T-cells were depleted, suggesting that both T-cell populations are essential for immunity against B-ALL cells. These results indicate that treating aged mice with recombinant IL-37 significantly boosts anti-leukemia T-cell-mediated immune responses.

### Recombinant IL-37 Treatment Improves the Efficacy of Aged Chimeric Antigen Receptor (CAR) T-Cells

Experiments were performed to determine whether rIL-37 treatment alters the efficacy of aged CAR T-cells. To this end, CD19-expressing CAR T-cells were engineered from T-cells isolated from aged (24 months old) mice and injected into aged (24 months old) recipient mice. On day 2 post-transplantation, mice were treated once weekly for 2 weeks with Control Ig or rIL-37. CAR T-cells were then purified from the spleen and stimulated with murine CD19-expressing B-ALL cells to determine the ex vivo production of IL-2 and IFN-γ. Consistent with the improvements in the function of aged endogenous T-cells, rIL-37 treatment also increased IL-2 and IFN-γ production from aged CD4+ and CD8+ CAR T-cells.

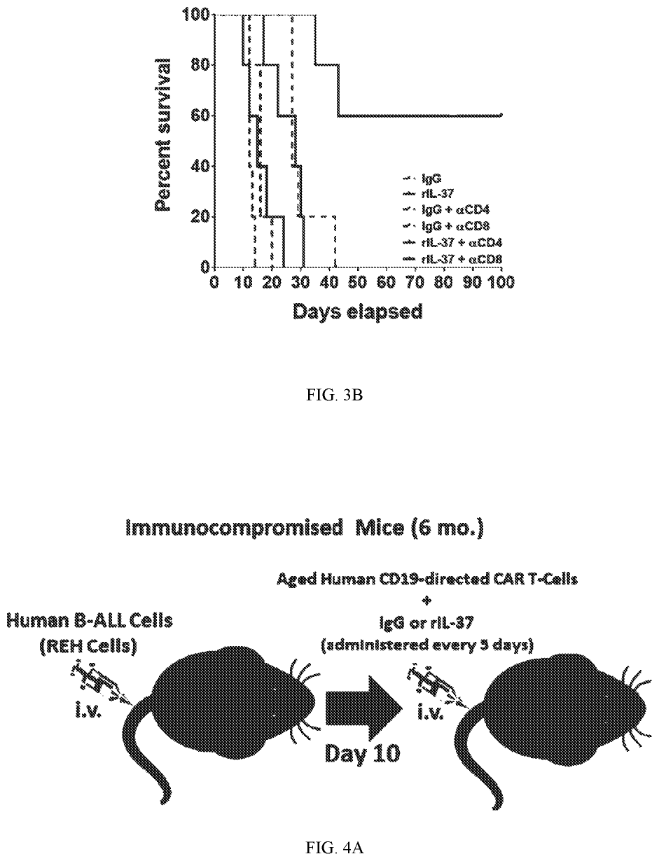

Experiments were performed to determine whether rIL-37 treatment impacts the efficacy of aged human CAR T-cells in vivo. Immunocompromised mice (6 months old) were transplanted with human B-ALL cells, and all mice injected with B-ALL cells exhibited signs of morbidity by day 7 post-transplantation. On day 10 post-transplantation of B-ALL cells, mice began receiving treatment with human CD19-directed CAR T-cells (generated from a 67-year-old donor) with or without the coadministration of rIL-37 (which continued weekly for the duration of the experiment; FIG. 4A). Treating mice with human CAR T-cells and control Ig resulted in 20% of mice surviving for greater than 3 months (FIG. 4B). When CAR T-cell therapy was combined with rIL-37 treatment, the 3-month survival of mice significantly increased to 60% (FIG. 4B). These experiments indicate IL-37 can rejuvenate the function of aged endogenous T-cells and boost the efficacy of aged CAR T-cells resulting in attenuated B-ALL pathogenesis.

### CD19 CAR+ IL-37 Armored CAR Sequence

CD19 CAR were used; however, it is contemplated that any CAR structure in combination with IL-37 CD19 CAR structure can be utilized. A CAR with CD28 as costimulatory domain, CD19 scFv sequence was derived from FMC63 clone reported in Nicholson I C et al. Mol Immunol. 1997, 34(16-17):1157-1165.

CD19 scFv (VH): 360 bp-120 AA
(SEQ ID NO: 1)
GAGGTGAAACTGCAGGAGTCAGGACCTGGCCTGGTGGCGC
CCTCACAGAGCCTGTCCGTCACATGCACTGTCTCAGGGGT
CTCATTACCCGACTATGGTGTAAGCTGGATTCGCCAGCCT
CCACGAAAGGGTCTGGAGTGGCTGGGAGTAATATGGGGTA
GTGAAACCACATACTATAATTCAGCTCTCAAATCCAGACT
GACCATCATCAAGGACAACTCCAAGAGCCAAGTTTTCTTA
AAAATGAACAGTCTGCAAACTGATGACACAGCCATTTACT
ACTGTGCCAAACATTATTACTACGGTGGTAGCTATGCTAT
GGACTACTGGGGTCAAGGAACCTCAGTCACCGTCTCCTCA
(SEQ ID NO: 2)
EVKLQESGPGLVAPSQSLSVTCTVSGVSLPDYGVSWIRQP
PRKGLEWLGVIWGSETTYYNSALKSRLTIIKDNSKSQVFL
KMNSLQTDDTAIYYCAKHYYYGGSYAMDYWGQGTSVTVSS
CD19 VL: 369 bp-123 AA
(SEQ ID NO: 3)
GACATCCAGATGACACAGACTACATCCTCCCTGTCTGCCT
CTCTGGGAGACAGAGTCACCATCAGTTGCAGGGCAAGTCA
GGACATTAGTAAATATTTAAATTGGTATCAGCAGAAACCA
GATGGAACTGTTAAACTCCTGATCTACCATACATCAAGAT
TACACTCAGGAGTCCCATCAAGGTTCAGTGGCAGTGGGTC
TGGAACAGATTATTCTCTCACCATTAGCAACCTGGAGCAA
GAAGATATTGCCACTTACTTTTGCCAACAGGGTAATACGC
TTCCGTACACGTTCGGAGGGGGGACTAAGTTGGAAATAAC
ACGGGCTGATGCTGCACCAACTGTATCCATCTTCCCACCA
TCCAGTAAT
(SEQ ID NO: 4)
DIQMTQTTSSLSASLGDRVTISCRASQDISKYLNWYQQKP
DGTVKLLIYHTSRLHSGVPSRFSGSGSGTDYSLTISNLEQ
EDIATYFCQQGNTLPYTFGGGTKLEITRADAAPTVSIFPP
SSN
Linker sequence (G4S)3-45 bp-15 AA
(SEQ ID NO: 5)
GGCGGGGGAGGATCAGGTGGTGGCGGTAGCGGTGGAGGTGGAAGT
(SEQ ID NO: 6)
GGGGSGGGGSGGGGS
CD19 scFv (VH) + linker + VL: 774 bp-258AA
(SEQ ID NO: 7)
GAGGTGAAACTGCAGGAGTCAGGACCTGGCCTGGTGGCGC
CCTCACAGAGCCTGTCCGTCACATGCACTGTCTCAGGGGT
CTCATTACCCGACTATGGTGTAAGCTGGATTCGCCAGCCT
CCACGAAAGGGTCTGGAGTGGCTGGGAGTAATATGGGGTA
GTGAAACCACATACTATAATTCAGCTCTCAAATCCAGACT
GACCATCATCAAGGACAACTCCAAGAGCCAAGTTTTCTTA
AAAATGAACAGTCTGCAAACTGATGACACAGCCATTTACT
ACTGTGCCAAACATTATTACTACGGTGGTAGCTATGCTAT
GGACTACTGGGGTCAAGGAACCTCAGTCACCGTCTCCTCA
GGCGGGGGAGGATCAGGTGGTGGCGGTAGCGGTGGAGGTG
GAAGTGACATCCAGATGACACAGACTACATCCTCCCTGTC
TGCCTCTCTGGGAGACAGAGTCACCATCAGTTGCAGGGCA
AGTCAGGACATTAGTAAATATTTAAATTGGTATCAGCAGA
AACCAGATGGAACTGTTAAACTCCTGATCTACCATACATC
AAGATTACACTCAGGAGTCCCATCAAGGTTCAGTGGCAGT
GGGTCTGGAACAGATTATTCTCTCACCATTAGCAACCTGG
AGCAAGAAGATATTGCCACTTACTTTTGCCAACAGGGTAA
TACGCTTCCGTACACGTTCGGAGGGGGGACTAAGTTGGAA
ATAACACGGGCTGATGCTGCACCAACTGTATCCATCTTCC
CACCATCCAGTAAT
(SEQ ID NO: 8)
EVKLQESGPGLVAPSQSLSVTCTVSGVSLPDYGVSWIRQP
PRKGLEWLGVIWGSETTYYNSALKSRLTIIKDNSKSQVFL
KMNSLQTDDTAIYYCAKHYYYGGSYAMDYWGQGTSVTVSS
GGGGSGGGGSGGGGSDIQMTQTTSSLSASLGDRVTISCRA
SQDISKYLNWYQQKPDGTVKLLIYHTSRLHSGVPSRFSGS
GSGTDYSLTISNLEQEDIATYFCQQGNTLPYTFGGGTKLE
ITRADAAPTVSIFPPSSN
Codon optimized CD19 scFv sequence for
humans: 774 bp-258 AA
(SEQ ID NO: 9)
GAGGTCAAGCTCCAAGAATCTGGGCCTGGTTTGGTCGCGC
CCTCTCAGTCTTTGTCCGTCACTTGTACTGTTTCCGGCGT
TTCTCTGCCCGATTACGGAGTCTCTTGGATACGGCAGCCC
CCACGAAAGGGGTTGGAGTGGTTGGGCGTTATATGGGGAT
CAGAAACAACGTATTACAACTCCGCGCTCAAGAGCAGACT
TACTATTATAAAGGATAACAGTAAATCACAGGTGTTCCTG
AAAATGAACTCTTTGCAAACCGATGATACGGCGATCTACT
ATTGTGCGAAGCACTATTACTACGGTGGTAGCTACGCGAT
GGACTATTGGGGCCAAGGGACGTCTGTCACAGTATCATCT
GGTGGAGGTGGGAGTGGAGGAGGCGGCAGTGGAGGCGGGG
GGAGTGACATCCAGATGACGCAGACGACTTCTTCACTCTC
TGCATCTTTGGGAGATCGGGTGACTATCAGTTGCAGGGCG
TCCCAGGACATATCAAAGTACCTTAACTGGTACCAGCAGA
AACCCGATGGGACAGTAAAACTTCTTATATATCATACTTC
TCGGCTGCATTCCGGTGTGCCATCTAGGTTTTCAGGTTCT
GGCTCTGGAACCGACTACTCCTTGACGATTTCTAACCTCG
AACAAGAGGACATAGCTACCTATTTTTGTCAGCAGGGAAA
CACTCTCCCGTACACGTTTGGAGGGGGAACTAAACTGGAG
ATCACGCGGGCTGACGCGGCTCCAACTGTGAGTATCTTCC
CACCGTCCTCAAAT
(SEQ ID NO: 10)
EVKLQESGPGLVAPSQSLSVTCTVSGVSLPDYGVSWIRQP
PRKGLEWLGVIWGSETTYYNSALKSRLTIIKDNSKSQVFL
KMNSLQTDDTAIYYCAKHYYYGGSYAMDYWGQGTSVTVSS
GGGGSGGGGSGGGGSDIQMTQTTSSLSASLGDRVTISCRA
SQDISKYLNWYQQKPDGTVKLLIYHTSRLHSGVPSRFSGS
GSGTDYSLTISNLEQEDIATYFCQQGNTLPYTFGGGTKLE
ITRADAAPTVSIFPPSSN
NheI restriction site
(SEQ ID NO: 11)
GCT AGC
encoding amino acids AR (alanine-arginine)
Human CD8 stalk region (codon optimized)-
138 bp 46 AA
(SEQ ID NO: 12)
ACCACTACCCCGGCCCCTAGGCCCCCTACTCCAGCGCCAA
CTATAGCATCACAGCCTTTGAGCTTGAGGCCCGAAGCTTG
CAGACCGGCGGCAGGGGGGGCTGTGCATACAAGGGGCCTC
GACTTTGCCTGCGACATC
(SEQ ID NO: 13)
TTTPAPRPPTPAPTIASQPLSLRPEACRPAAGGAVHTRGLDFACDI
CD28 + CD3 zeta + Stop codon: 618 bp 206 AA
(SEQ ID NO: 14)
GATAATGAGAAGAGCAATGGAACCATTATCCATGTGAAAG
GGAAACACCTTTGTCCAAGTCCCCTATTTCCCGGACCTTC
TAAGCCCTTTTGGGTGCTGGTGGTGGTTGGTGGAGTCCTG
GCTTGCTATAGCTTGCTAGTAACAGTGGCCTTTATTATTT
TCTGGGTGAGGAGTAAGAGGAGCAGGCTCCTGCACAGTGA
CTACATGAACATGACTCCCAGGAGGCCTGGGCCAACCCGC
AAGCATTACCAGCCCTATGCCCCACCACGCGACTTCGCAG
CCTATCGCTCCAGCAGGAGCGCAGACGCTCCCGCGTACCA
GCAGGGCCAGAACCAGCTCTATAACGAGCTCAATCTAGGA
CGAAGAGAGGAGTACGATGTTTTGGACAAGAGACGTGGCC
GGGACCCTGAGATGGGAGGCAAGCCGAGAAGGAAGAACCC
TCAGGAAGGCCTGTACAATGAACTGCAGAAAGATAAGATG
GCGGAGGCCTACAGTGAGATTGGGATGAAAGGCGAGCGCC
GGAGGGGCAAGGGGCACGATGGCCTTTACCAGGGTCTCAG
TACAGCCACCAAGGACACCTACGACGCCCTTCACATGCAG
GCCCTGCCTCCTCGCTGA
(SEQ ID NO: 15)
DNEKSNGTIIHVKGKHLCPSPLFPGPSKPFWVLVVVGGVL
ACYSLLVTVAFIIFWVRSKRSRLLHSDYMNMTPRRPGPTR
KHYQPYAPPRDFAAYRSSRSADAPAYQQGQNQLYNELNLG
RREEYDVLDKRRGRDPEMGGKPRRKNPQEGLYNELQKDKM
AEAYSEIGMKGERRRGKGHDGLYQGLSTATKDTYDALHMQ
ALPPR
CD19 CAR: IL-2 signal peptide
Asc restriction site
CD19 scFv + Nhe1 restriction site
CD8 alpha + CD28 + CD3 zeta + stop codon
(SEQ ID NO: 16)
ATGTACAGGATGCAACTCCTGTCTTGCATTGCACTAAGTC
TTGCACTTGTCACGAATTCGGGCGCGCCTGAGGTCAAGCT
CCAAGAATCTGGGCCTGGTTTGGTCGCGCCCTCTCAGTCT
TTGTCCGTCACTTGTACTGTTTCCGGCGTTTCTCTGCCCG
ATTACGGAGTCTCTTGGATACGGCAGCCCCCACGAAAGGG
GTTGGAGTGGTTGGGCGTTATATGGGGATCAGAAACAACG
TATTACAACTCCGCGCTCAAGAGCAGACTTACTATTATAA
AGGATAACAGTAAATCACAGGTGTTCCTGAAAATGAACTC
TTTGCAAACCGATGATACGGCGATCTACTATTGTGCGAAG
CACTATTACTACGGTGGTAGCTACGCGATGGACTATTGGG
GCCAAGGGACGTCTGTCACAGTATCATCTGGTGGAGGTGG
GAGTGGAGGAGGCGGCAGTGGAGGCGGGGGGAGTGACATC
CAGATGACGCAGACGACTTCTTCACTCTCTGCATCTTTGG
GAGATCGGGTGACTATCAGTTGCAGGGCGTCCCAGGACAT
ATCAAAGTACCTTAACTGGTACCAGCAGAAACCCGATGGG
ACAGTAAAACTTCTTATATATCATACTTCTCGGCTGCATT
CCGGTGTGCCATCTAGGTTTTCAGGTTCTGGCTCTGGAAC
CGACTACTCCTTGACGATTTCTAACCTCGAACAAGAGGAC
ATAGCTACCTATTTTTGTCAGCAGGGAAACACTCTCCCGT
ACACGTTTGGAGGGGGAACTAAACTGGAGATCACGCGGGC
TGACGCGGCTCCAACTGTGAGTATCTTCCCACCGTCCTCA
AATGCTAGCACCACTACCCCGGCCCCTAGGCCCCCTACTC
CAGCGCCAACTATAGCATCACAGCCTTTGAGCTTGAGGCC
CGAAGCTTGCAGACCGGCGGCAGGGGGGGCTGTGCATACA
AGGGGCCTCGACTTTGCCTGCGACATCGATAATGAGAAGA
GCAATGGAACCATTATCCATGTGAAAGGGAAACACCTTTG
TCCAAGTCCCCTATTTCCCGGACCTTCTAAGCCCTTTTGG
GTGCTGGTGGTGGTTGGTGGAGTCCTGGCTTGCTATAGCT
TGCTAGTAACAGTGGCCTTTATTATTTTCTGGGTGAGGAG
TAAGAGGAGCAGGCTCCTGCACAGTGACTACATGAACATG
ACTCCCAGGAGGCCTGGGCCAACCCGCAAGCATTACCAGC
CCTATGCCCCACCACGCGACTTCGCAGCCTATCGCTCCAG
CAGGAGCGCAGACGCTCCCGCGTACCAGCAGGGCCAGAAC
CAGCTCTATAACGAGCTCAATCTAGGACGAAGAGAGGAGT
ACGATGTTTTGGACAAGAGACGTGGCCGGGACCCTGAGAT
GGGAGGCAAGCCGAGAAGGAAGAACCCTCAGGAAGGCCTG
TACAATGAACTGCAGAAAGATAAGATGGCGGAGGCCTACA
GTGAGATTGGGATGAAAGGCGAGCGCCGGAGGGGCAAGGG
GCACGATGGCCTTTACCAGGGTCTCAGTACAGCCACCAAG
GACACCTACGACGCCCTTCACATGCAGGCCCTGCCTCCTC
GCTGA
(SEQ ID NO: 17)
MYRMQLLSCIALSLALVTNSGAPEVKLQESGPGLVAPSQS
LSVTCTVSGVSLPDYGVSWIRQPPRKGLEWLGVIWGSETT
YYNSALKSRLTIIKDNSKSQVFLKMNSLQTDDTAIYYCAK
HYYYGGSYAMDYWGQGTSVTVSSGGGGSGGGGSGGGGSDI
QMTQTTSSLSASLGDRVTISCRASQDISKYLNWYQQKPDG
TVKLLIYHTSRLHSGVPSRFSGSGSGTDYSLTISNLEQED
IATYFCQQGNTLPYTFGGGTKLEITRADAAPTVSIFPPSS
NASTTTPAPRPPTPAPTIASQPLSLRPEACRPAAGGAVHT
RGLDFACDIDNEKSNGTIIHVKGKHLCPSPLFPGPSKPFW
VLVVVGGVLACYSLLVTVAFIIFWVRSKRSRLLHSDYMNM
TPRRPGPTRKHYQPYAPPRDFAAYRSSRSADAPAYQQGQN
QLYNELNLGRREEYDVLDKRRGRDPEMGGKPRRKNPQEGL
YNELQKDKMAEAYSEIGMKGERRRGKGHDGLYQGLSTATK
DTYDALHMQALPPR
IL-37 sequence (UniPROT): 218 AA-654 bp
(codon optimized for homo sapiens)
(SEQ ID NO: 18)
MSFVGENSGVKMGSEDWEKDEPQCCLEDPAGSPLEPGPSL
PTMNFVHTSPKVKNLNPKKFSIHDQDHKVLVLDSGNLIA
VPDKNYIRPEIFFALASSLSSASAEKGSPILLGVSKGEFC
LYCDKDKGQSHPSLQLKKEKLMKLAAQKESARRPFIFYRA
QVGSWNMLESAAHPGWFICTSCNCNEPVGVTDKFENRKHI
EFSFQPVCKAEMSPSEVSD
(SEQ ID NO: 19)
ATGAGTTTCGTCGGCGAAAACAGCGGAGTAAAAATGGGAA
GCGAAGATTGGGAAAAGGACGAGCCGCAATGCTGTTTGGA
AGATCCGGCTGGCTCCCCTCTGGAACCAGGTCCAAGCCTT
CCGACGATGAATTTCGTTCACACCAGTCCGAAAGTAAAGA
ATCTTAATCCCAAGAAATTTTCAATCCACGACCAAGACCA
CAAGGTTCTCGTACTTGATTCTGGAAACCTTATAGCGGTA
CCCGATAAGAACTATATTAGGCCAGAGATTTTCTTTGCGT
TGGCCTCATCACTGAGCAGTGCTTCTGCTGAAAAGGGGAG
CCCAATCCTCCTGGGCGTGTCAAAGGGCGAGTTCTGCCTC
TACTGTGATAAAGATAAGGGCCAGTCTCATCCATCACTTC
AGTTGAAAAAGGAAAAACTGATGAAACTCGCGGCTCAGAA
AGAGAGCGCCCGGCGGCCATTTATATTTTATCGGGCACAA
GTTGGTAGCTGGAATATGCTCGAGAGCGCAGCACATCCCG
GATGGTTCATTTGTACTTCATGTAATTGCAACGAGCCCGT
GGGTGTCACGGATAAATTTGAGAACCGGAAACATATCGAA
TTTTCTTTCCAGCCTGTATGCAAGGCAGAGATGTCCCCGA
GTGAGGTAAGTGAC

### Armored CAR

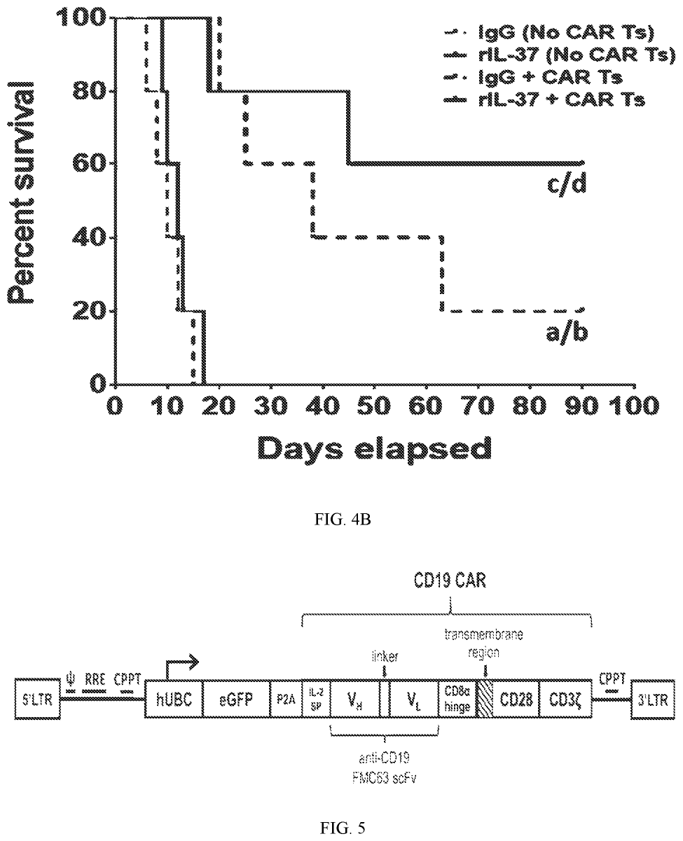

Bicistronic constructs (see FIG. 5) utilizing a P2A self-cleaving sequence to allow for dual expression of both the CD19-CAR and IL-37 sequences:

(SEQ ID NO: 20)
GGATCTGGAGCAACAAACTTCTCACTACTCAAACAAGCAG
GTGACGTGGAGGAGAATCCCGGGCCT
(SEQ ID NO: 21)
GSGATNFSLLKQAGDVEENPGP

In certain embodiments, this disclosure relates to vectors encoding a chimeric antigen receptor and IL-37. In certain embodiments, the chimeric antigen receptor and IL-37 are separated by a self-cleaving spacer, GSGATNFSLLKQAGDVEENPGP (SEQ ID NO: 21). In certain embodiments, IL-37 is connected to the N-terminal of the self-cleaving spacer and the chimeric antigen receptor is connected to the C-terminal of the self-cleaving spacer, and IL-37 has the amino acid sequence of

MSFVGENSGVKMGSEDWEKDEPQCCLEDPAGSPLEPGPSLPTMNFVHTSPKVKN LNPKKFSIHDQDHKVLVLDSGNLIAVPDKNYIRPEIFFALASSLSSASAEKGSPILLGVSK GEFCLYCDKDKGQSUPSLQLKKEKLMKLAAQKESARRPFIFYRAQVGSWNMLESAAHP GWFICTSCNCNEPVGVTDKFENRKHIEFSFQPVCKAEMSPSEVSD (SEQ ID NO: 18) or variants thereof, e.g., have greater than 60, 70, 80, 85, 90, 95, 98 or 99% sequence identity.

### Lentiviral Vector Production (CAR-T Generation)

High-titer, recombinant, self-inactivating (SIN) HIV lentiviral vector was produced using a four-plasmid system. Briefly, the expression plasmid encoding the CD19-CAR construct as well as packaging plasmids containing the gag, pol, and envelope (VSV-G) genes were transiently transfected into HEK-293T cells by calcium phosphate transfection. Cells were cultured in Dulbecco's modified essential medium (DMEM, Thermo Fisher Scientific) supplemented with 10% FBS and 1% penicillin-streptomycin. Twenty-four hours after transfection, the cell culture medium was replaced with fresh medium. At 48, 72, and 96 hours the viral supernatant was collected, filtered through a 0.22 m filter and stored at −80° C. After the final virus collection, the supernatant was pooled and concentrated overnight via centrifugation at 10,000 g at 4° C. Pelleted virus was then resuspended in StemPro media (Thermo Fisher Scientific). Titer of the concentrated virus was found to be ˜1×10⁸ transducing units (TU)/mL, i.e., on HEK-293T cells using quantitative polymerase chain reaction.

### Lentiviral Transduction of T-Cells (CAR-T Generation)

Human T-cells were isolated from cryopreserved peripheral blood mononuclear cells (PBMCs) purchased from AllCells™ or isolated from mice using magnetic-activated cell sorting T-cells were activated with anti-CD3/CD28 Dynabeads' for 24 hours prior to transduction. Transduction of recombinant HIV lentiviral particles was carried out by incubating cells with the CD19-CAR encoding lentiviral vector in complete medium supplemented with 8 μg/ml polybrene (EMD Millipore, Billerica, MA) at a multiplicity of infection (MOI) of 25. Eighteen hours after transduction, media was replaced. The transduced cells were then cultured for at least 5 days prior to use in experiments, with media replacement occurring every 3 days.

What is claimed is:

1. A method of treating cancer comprising administering an effective amount of T cells expressing a cancer targeting chimeric antigen receptor and administering a vector encoding a recombinant IL-37 protein to a subject in need thereof.

2. The method of claim 1, wherein the subject is over 55 or 65 years of age.

3. The method of claim 1, wherein the subject is medically immune suppressed.

4. The method of claim 1, wherein the vector encoding IL-37 is administered more than one day before administration of the T cells.

5. The method of claim 1, wherein the vector encoding IL-37 is administered in combination with another anticancer agent.

6. The method of claim 5, wherein the anticancer agent is a checkpoint inhibitor, an anti-PD-1, anti-PD-L1 anti-CTLA4 antibody or combinations thereof.

7. The method of claim 1, wherein the chimeric antigen receptor specifically binds CD138, CD19, immunoglobulin kappa (Ig-Kappa) or B-cell maturation antigen (BCMA).

8. The method of claim 1, wherein the vector encoding IL-37 is administered to a subject with a lymphodepleted environment due to prior or concurrent administration of a lymphodepleting agent.

9. The method of claim 8, wherein the of lymphodepleting agent is cyclophosphamide, fludarabine, or combination thereof.

10. A method of treating a hematological malignancy comprising administering an effective amount of T cells expressing a cancer targeting chimeric antigen receptor and expressing a recombinant IL-37 protein to a subject in need thereof.

11. The method of claim 10, wherein the subject is over 55 or 65 years of age.

12. The method of claim 10, wherein the cancer targeting chimeric antigen receptor and the recombinant IL-37 protein are expressed in a single vector.

13. The method of claim 10, wherein the cancer targeting chimeric antigen receptor and the recombinant IL-37 protein are expressed in separate vectors.

14. The method of claim 10, wherein the T cells are administered in combination with another anticancer agent.

15. A vector encoding a chimeric antigen receptor and IL-37.

16. The vector or claim 15, wherein the chimeric antigen receptor and IL-37 are separated by a self-cleaving spacer, GSGATNFSLLKQAGDVEENPGP (SEQ ID NO: 21).

17. The vector of claim 16, wherein the IL-37 is connected to the N-terminal of the self-cleaving spacer and the chimeric antigen receptor is connected to the C-terminal of the self-cleaving spacer.

18. The vector of claim 17, wherein IL-37 has the amino acid sequence of
(SEQ ID NO: 18)
MSFVGENSGVKMGSEDWEKDEPQCCLEDPAGSPLEPGPSL
PTMNFVHTSPKVKNLNPKKFSIHDQDHKVLVLDSGNLIAV
PDKNYIRPEIFFALASSLSSASAEKGSPILLGVSKGEFCL
YCDKDKGQSHPSLQLKKEKLMKLAAQKESARRPFIFYRAQ
VGSWNMLESAAHPGWFICTSCNCNEPVGVTDKFENRKHIE
FSFQPVCKAEMSPSEVSD


SEQUENCE LISTING (21 sequences)

SEQ ID NO: 1 - DNA, 360 bp, Artificial
  Synthetic construct
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 2 - protein, 120 aa, Artificial
  Synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - DNA, 369 bp, Artificial
  Synthetic construct
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaat

SEQ ID NO: 4 - protein, 123 aa, Artificial
  Synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSN

SEQ ID NO: 5 - DNA, 45 bp, Artificial
  Synthetic construct
ggcgggggaggatcaggtggtggcggtagcggtggaggtggaagt

SEQ ID NO: 6 - protein, 15 aa, Artificial
  Synthetic construct
GGGGSGGGGSGGGGS

SEQ ID NO: 7 - DNA, 774 bp, Artificial
  Synthetic construct
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggcgggggaggatcaggtggtggcggtagcggtggaggtggaagtgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacacgggctgatgctgcaccaactgtatccatcttcccaccatccagtaat

SEQ ID NO: 8 - protein, 258 aa, Artificial
  Synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSN

SEQ ID NO: 9 - DNA, 774 bp, Artificial
  Synthetic construct
gaggtcaagctccaagaatctgggcctggtttggtcgcgccctctcagtctttgtccgtcacttgtactgtttccggcgtttctctgcccgattacggagtctcttggatacggcagcccccacgaaaggggttggagtggttgggcgttatatggggatcagaaacaacgtattacaactccgcgctcaagagcagacttactattataaaggataacagtaaatcacaggtgttcctgaaaatgaactctttgcaaaccgatgatacggcgatctactattgtgcgaagcactattactacggtggtagctacgcgatggactattggggccaagggacgtctgtcacagtatcatctggtggaggtgggagtggaggaggcggcagtggaggcggggggagtgacatccagatgacgcagacgacttcttcactctctgcatctttgggagatcgggtgactatcagttgcagggcgtcccaggacatatcaaagtaccttaactggtaccagcagaaacccgatgggacagtaaaacttcttatatatcatacttctcggctgcattccggtgtgccatctaggttttcaggttctggctctggaaccgactactccttgacgatttctaacctcgaacaagaggacatagctacctatttttgtcagcagggaaacactctcccgtacacgtttggagggggaactaaactggagatcacgcgggctgacgcggctccaactgtgagtatcttcccaccgtcctcaaat

SEQ ID NO: 10 - protein, 258 aa, Artificial; identical to SEQ ID NO: 8
  Synthetic construct

SEQ ID NO: 11 - DNA, 6 bp, Artificial
  Synthetic construct
gctagc

SEQ ID NO: 12 - DNA, 138 bp, Artificial
  Synthetic construct
accactaccccggcccctaggccccctactccagcgccaactatagcatcacagcctttgagcttgaggcccgaagcttgcagaccggcggcagggggggctgtgcatacaaggggcctcgactttgcctgcgacatc

SEQ ID NO: 13 - protein, 46 aa, Artificial
  Synthetic construct
TTTPAPRPPTPAPTIASQPLSLRPEACRPAAGGAVHTRGLDFACDI

SEQ ID NO: 14 - DNA, 618 bp, Artificial
  Synthetic construct
gataatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactcccaggaggcctgggccaacccgcaagcattaccagccctatgccccaccacgcgacttcgcagcctatcgctccagcaggagcgcagacgctcccgcgtaccagcagggccagaaccagctctataacgagctcaatctaggacgaagagaggagtacgatgttttggacaagagacgtggccgggaccctgagatgggaggcaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctga

SEQ ID NO: 15 - protein, 205 aa, Artificial
  Synthetic construct
DNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 1605 bp, Artificial
  Synthetic construct
atgtacaggatgcaactcctgtcttgcattgcactaagtcttgcacttgtcacgaattcgggcgcgcctgaggtcaagctccaagaatctgggcctggtttggtcgcgccctctcagtctttgtccgtcacttgtactgtttccggcgtttctctgcccgattacggagtctcttggatacggcagcccccacgaaaggggttggagtggttgggcgttatatggggatcagaaacaacgtattacaactccgcgctcaagagcagacttactattataaaggataacagtaaatcacaggtgttcctgaaaatgaactctttgcaaaccgatgatacggcgatctactattgtgcgaagcactattactacggtggtagctacgcgatggactattggggccaagggacgtctgtcacagtatcatctggtggaggtgggagtggaggaggcggcagtggaggcggggggagtgacatccagatgacgcagacgacttcttcactctctgcatctttgggagatcgggtgactatcagttgcagggcgtcccaggacatatcaaagtaccttaactggtaccagcagaaacccgatgggacagtaaaacttcttatatatcatacttctcggctgcattccggtgtgccatctaggttttcaggttctggctctggaaccgactactccttgacgatttctaacctcgaacaagaggacatagctacctatttttgtcagcagggaaacactctcccgtacacgtttggagggggaactaaactggagatcacgcgggctgacgcggctccaactgtgagtatcttcccaccgtcctcaaatgctagcaccactaccccggcccctaggccccctactccagcgccaactatagcatcacagcctttgagcttgaggcccgaagcttgcagaccggcggcagggggggctgtgcatacaaggggcctcgactttgcctgcgacatcgataatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactcccaggaggcctgggccaacccgcaagcattaccagccctatgccccaccacgcgacttcgcagcctatcgctccagcaggagcgcagacgctcccgcgtaccagcagggccagaaccagctctataacgagctcaatctaggacgaagagaggagtacgatgttttggacaagagacgtggccgggaccctgagatgggaggcaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctga

SEQ ID NO: 17 - protein, 534 aa, Artificial
  Synthetic construct
MYRMQLLSCIALSLALVTNSGAPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITRADAAPTVSIFPPSSNASTTTPAPRPPTPAPTIASQPLSLRPEACRPAAGGAVHTRGLDFACDIDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 218 aa, Artificial
  Synthetic construct
MSFVGENSGVKMGSEDWEKDEPQCCLEDPAGSPLEPGPSLPTMNFVHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 19 - DNA, 653 bp, Artificial
  Synthetic construct
tgagtttcgtcggcgaaaacagcggagtaaaaatgggaagcgaagattgggaaaaggacgagccgcaatgctgtttggaagatccggctggctcccctctggaaccaggtccaagccttccgacgatgaatttcgttcacaccagtccgaaagtaaagaatcttaatcccaagaaattttcaatccacgaccaagaccacaaggttctcgtacttgattctggaaaccttatagcggtacccgataagaactatattaggccagagattttctttgcgttggcctcatcactgagcagtgcttctgctgaaaaggggagcccaatcctcctgggcgtgtcaaagggcgagttctgcctctactgtgataaagataagggccagtctcatccatcacttcagttgaaaaaggaaaaactgatgaaactcgcggctcagaaagagagcgcccggcggccatttatattttatcgggcacaagttggtagctggaatatgctcgagagcgcagcacatcccggatggttcatttgtacttcatgtaattgcaacgagcccgtgggtgtcacggataaatttgagaaccggaaacatatcgaattttctttccagcctgtatgcaaggcagagatgtccccgagtgaggtaagtgac

SEQ ID NO: 20 - DNA, 66 bp, Artificial
  Synthetic construct
ggatctggagcaacaaacttctcactactcaaacaagcaggtgacgtggaggagaatcccgggcct

SEQ ID NO: 21 - protein, 22 aa, Artificial
  Synthetic construct
GSGATNFSLLKQAGDVEENPGP